(19) United States
(12) Patent Application Publication
(10) Pub. No.: US 2023/0255823 A1
(43) Pub. Date: Aug. 17, 2023

(54) VITRECTOR WITH EVERTING SEAL

(71) Applicant: JOHNSON & JOHNSON SURGICAL VISION, INC., Irvine, CA (US)

(72) Inventors: Ilya Sitnitsky, Nahariya (IL); Alexander Shechtman, Haifa (IL); Assaf Govari, Haifa (IL); Stanislav Katzir, Hadera (IL); Elad Avraham Diukman, Haifa (IL)

(21) Appl. No.: 18/098,144

(22) Filed: Jan. 18, 2023

Related U.S. Application Data
(60) Provisional application No. 63/309,766, filed on Feb. 14, 2022.

(51) Int. Cl. A61F 9/007

(52) U.S. Cl. CPC A61F 9/00763

(57) ABSTRACT

A medical device having a housing, an annular fixture contained in the housing and having a central opening, a needle having a proximal end contained within the central opening of the annular fixture and a distal end protruding out of the housing, a motion assembly, which is configured to move the needle longitudinally back and forth relative to the annular fixture and the housing, and a flexible diaphragm comprising a peripheral part that seals externally around the annular fixture and an inner sleeve that seals around the proximal end of the needle, whereby movement of the needle back and forth causes the sleeve to evert and invert within the central opening while maintaining a vacuum seal between the annular fixture and the needle.

### CROSS-REFERENCE TO RELATED APPLICATIONS

This application claims the benefit of U.S. Provisional Patent Application 63/309,766, filed Feb. 14, 2022, whose disclosure is incorporated herein by reference.

### FIELD OF THE DISCLOSURE

The present disclosure relates generally to medical devices, and particularly to devices and methods for ophthalmic surgery.

### BACKGROUND OF THE DISCLOSURE

A vitrector is used in eye surgery to remove vitreous humor from the eye. The removal is based on back-and-forth motion of a cutting needle within an external casing, which is inserted into the eye. Tissue is sucked into a window at the end of the casing and is cut by the needle. The tissue fragments are then removed by suction through the cutting needle.

In examples of the present disclosure, the cutting needle of a vitrector moves back-and-forth longitudinally within its casing in a reciprocal motion at high frequency. This high frequency motion, if not carefully controlled, may be accompanied by lateral vibrations that can diminish the ability of the physician to properly control the vitrector and can cause harm to the eye. Tight dimensional tolerances are desirable to ensure that the needle is straight. Any deviation from a straight orientation exacerbates wear and increases the possibility of vibrations.

Examples of the present disclosure address this problem using a diaphragm with an internal sleeve at the center of the diaphragm to support the needle at its proximal end. The diaphragm is mounted on an annular fixture through which the needle extends. The end of the sleeve fits tightly around the needle and creates a vacuum seal. Back-and-forth motion of the needle is accompanied by eversion and inversion of the sleeve portion of the diaphragm relative to the inner surface of the annular fixture. There is no need for the needle to slide through the seal, and there is thus no relative motion between the needle and the sleeve.

The use of this sort of diaphragm with an everting sleeve has a number of advantages, including low friction, relaxed tolerance to variation in needle angle, provides a tight seal around the needle so that suction can be maintained through the needle, and simple, reliable assembly.

The present disclosure will be more fully understood from the following detailed description of the examples thereof, taken together with the drawings in which:

### BRIEF DESCRIPTION OF THE DRAWINGS

FIG. 1 is a schematic pictorial illustration of a vitrectomy device in accordance with an example of the present disclosure;

FIG. 2 is a schematic internal view of a vitrectomy device in accordance with an example of the disclosure;

FIG. 3 is a schematic internal view of a vitrectomy device in accordance with an example of the disclosure;

FIG. 4 is a schematic, sectional detail view along a longitudinal axis of a casing, showing an annular fixture, a needle, a diaphragm, and associated components of a vitrectomy device, in accordance with an example of the disclosure; and

FIGS. 5A and 5B are schematic sectional views along a longitudinal axis of a needle and a diaphragm, showing inversion (FIG. 5A) and eversion (FIG. 5B) of a 64 as the needle moves back and forth, in accordance with an example of the disclosure.

### DETAILED DESCRIPTION OF EXAMPLES

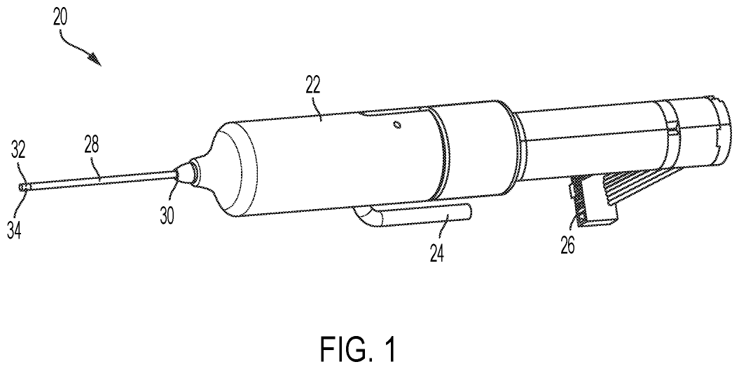

FIG. 1 is a schematic pictorial illustration of a vitrectomy device 20, in accordance with an example of the disclosure. Device 20 comprises a housing 22, which an ophthalmic surgeon holds in his or her hand in order to perform a vitrectomy procedure. The components contained inside housing 22 are shown in the figures that follow. Housing 22 comprises connectors, such as a vacuum connector 24 and an electrical connector 26, which connect to the components inside the housing.

A tubular casing 28 is attached at its proximal tip 30 to housing 22 and has a distal tip 32 configured for insertion into the eye of a patient. A window 34 opens radially in the distal tip of the tubular casing. A needle moves back and forth within casing 22, as shown in the figures that follow.

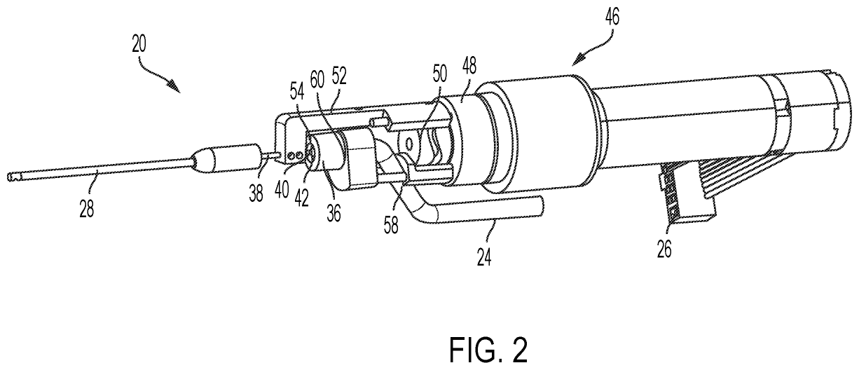

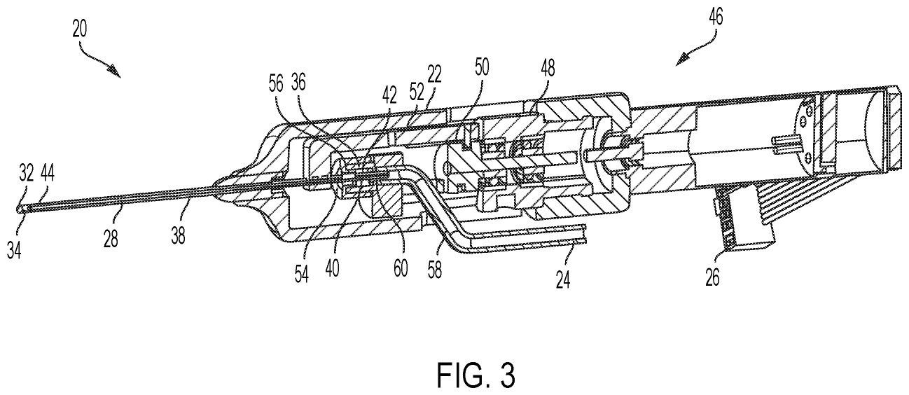

FIGS. 2 and 3 are schematic internal views of device 20, in accordance with an example of the disclosure. FIG. 2 is a pictorial view of the internal components of device 20, while FIG. 3 is a sectional view along a longitudinal axis of the device 20.

An annular fixture 36 is contained in and mounted fixedly within housing 22. A needle 38 has a proximal end 40 that is contained within a central opening 42 of annular fixture 36 and a distal end 44 that protrudes out of housing 22 and is contained within tubular casing 28. Needle 38 is mounted so that distal end 44 moves back and forth across window 34, thereby cutting tissue that is drawn into the window.

A motion assembly 46 moves needle 38 longitudinally back and forth relative to annular fixture 36 and to housing 22. In the pictured example, motion assembly 46 comprises a motor 48 and a reciprocating ring 50 connected to the shaft of the motor. A motion arm 52 is coupled with reciprocating ring 50, which drives arm 52 back and forth longitudinally in a reciprocal motion. The distal end of motion arm 52 couples with needle 38 and thus moves needle 38 back and forth within casing 28. These elements of motion assembly 46 are shown by way of example, and other mechanical arrangements can alternatively be used to impart the desired longitudinal reciprocating motion to needle 38. All such alternative implementations are considered to be within the scope of the present disclosure.

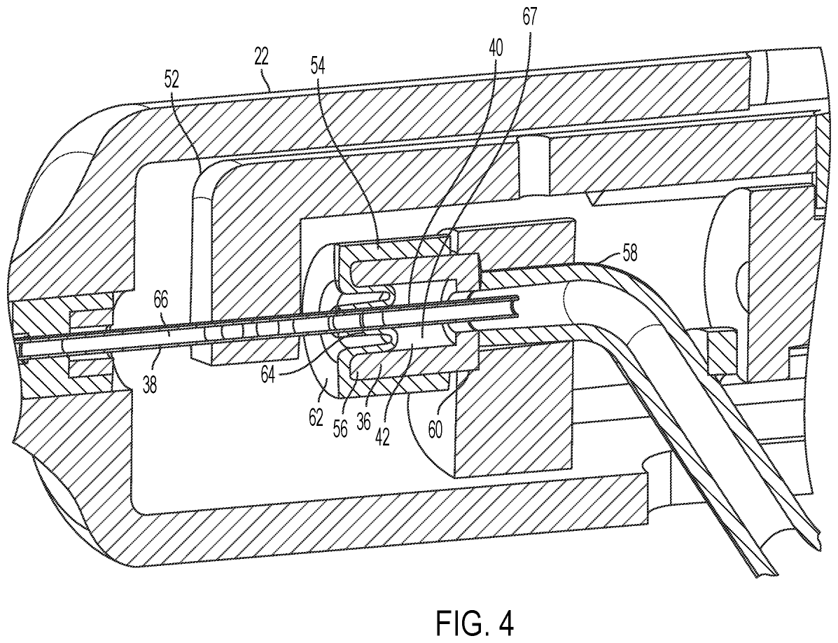

A flexible diaphragm 54 creates a seal between a distal side 56 of annular fixture 36 and needle 38, while permitting the needle to move longitudinally back and forth into and out of central opening 42 of the annular fixture 36. Diaphragm 54 comprises a flexible polymer, such as silicone or a suitable thermoplastic, such as polyurethane. An aspiration tube 58 is sealed to a proximal side 60 of annular fixture 36, thereby creating a sealed chamber within central opening 42 of annular fixture 36 (as shown in FIG. 4). Proximal end 40 of needle 38 moves back and forth within this chamber. Details of the structure and functionality of the diaphragm and the sealed chamber are illustrated in the figures that follow.

FIG. 4 is a schematic, sectional detail view along a longitudinal axis of casing 22, showing annular fixture 36, needle 38, diaphragm 54, and associated components of device 20, in accordance with an example of the disclosure. Diaphragm 54 comprises a peripheral part 62, which seals externally around distal side 56 of annular fixture 36, and an inner sleeve 64, which seals around proximal end 40 of needle 38. Movement of needle 38 back and forth causes sleeve 64 to evert and invert within central opening 42 while maintaining a vacuum seal between annular fixture 36 and needle 38.

Needle 38 contains a lumen 66, which opens at both the distal and proximal ends of the needle. Aspiration tube 58 is coupled in fluid communication with proximal end 40 of needle and thus provides suction via lumen 66 through distal end 44 of needle 38, to aspirate fragments of tissue that are drawn in through window 34 of casing 28. Aspiration tube 58, however, is static. Needle 38 moves within central opening 42 relative to the distal end of aspiration tube 58 without leakage or loss of vacuum suction. As noted earlier, the combination of diaphragm 54 at distal side 56 of annular fixture 36 with the seal between aspiration tube 58 and proximal side 60 of annular fixture 36 creates a sealed chamber 67 within central opening 42 of the annular fixture. Eversion and inversion of sleeve allows proximal end 40 of needle 38 to move longitudinally within the sealed chamber relative to aspiration tube 58.

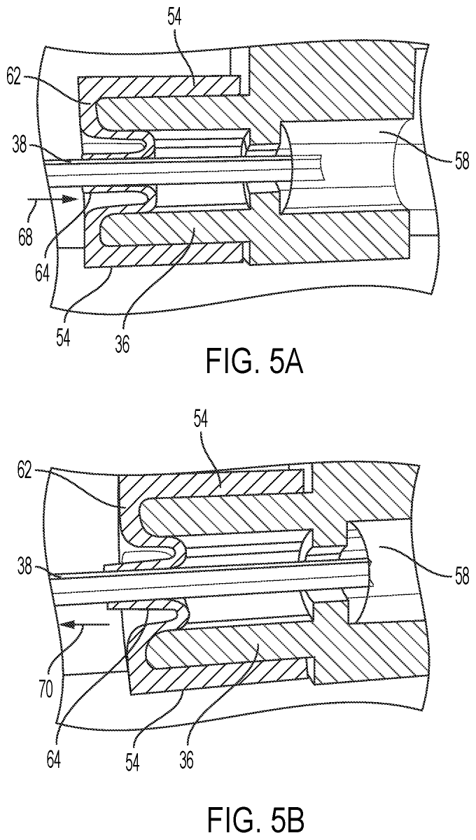

FIGS. 5A and 5B are schematic sectional views along a longitudinal axis of needle 38 and diaphragm 54, showing inversion (FIG. 5A) and eversion (FIG. 5B) of sleeve 64 as the needle moves back and forth, in accordance with an example of the disclosure. Sleeve 64 seals against the outer surface of needle 38, and there is no relative motion between the needle and the sleeve. In FIG. 5A, needle 38 is drawn to the right, into annular fixture 36, as indicated by an arrow 68, and sleeve 64 accordingly inverts into the central opening of the annular fixture 36. In FIG. 5B, needle is driven to the left, as indicated by an arrow 70, and sleeve 64 everts out of the annular fixture 36.

### EXAMPLES

### Example 1

A medical device (20) includes a housing (22), an annular fixture (36) contained in the housing and having a central opening; a needle (38) having a proximal end (40) contained within the central opening (42) of the annular fixture and a distal end (44) protruding out of the housing; a motion assembly (46), which is configured to move the needle longitudinally back and forth relative to the annular fixture and the housing; and a flexible diaphragm (54) comprising a peripheral part (62) that seals externally around the annular fixture and an inner sleeve (64) that seals around the proximal end of the needle, whereby movement of the needle back and forth causes the sleeve to evert and invert within the central opening while maintaining a vacuum seal between the annular fixture and the needle.

### Example 2

The device according to example 1, further including a tubular casing (28) having a proximal tip (30) coupled with the housing and a distal tip (32) configured for insertion into a body of a patient, with a window (34) opening radially in the distal tip of the tubular casing, wherein the needle is contained within the tubular casing and mounted so that the distal end of the needle moves back and forth across the window, thereby enabling tissue to be cut within the window.

### Example 3

The device according to any of the examples 1 through 2, wherein the motion assembly includes a motor (48), which is configured to move the needle back and forth in a reciprocating motion.

### Example 4

The device according to any of the examples 1 through 3, wherein the needle contains a lumen (66), which opens at both the distal and proximal ends, and wherein the device comprises an aspiration tube (58), which is coupled in fluid communication with the proximal end of the needle so as to provide suction via the lumen through the distal end of the needle.

### Example 5

The device according to any of the examples 1 through 4, wherein the peripheral part (62) of the diaphragm (54) seals externally around a distal side of the annular fixture, while the aspiration tube is sealed to a proximal side of the annular fixture, thereby creating a sealed chamber within the central opening of the annular fixture, such that the proximal end of the needle moves longitudinally within the sealed chamber relative to the aspiration tube.

### Example 6

A method for operating a device, includes providing a medical device (20), the device having a housing, an annular fixture contained in the housing and having a central opening, a needle having a proximal end contained within the central opening of the annular fixture and a distal end protruding out of the housing, a motion assembly, which is configured to move the needle longitudinally back and forth relative to the annular fixture and the housing, and a flexible diaphragm comprising a peripheral part that seals externally around the annular fixture and an inner sleeve that seals around the proximal end of the needle; and moving the needle back and forth within the annular fixture thereby causing the sleeve to evert and invert within the central opening while maintaining a vacuum seal between the annular fixture and the needle.

It will be appreciated that the examples described above are cited by way of example, and that the present disclosure is not limited to what has been particularly shown and described hereinabove. Rather, the scope of the present disclosure includes both combinations and subcombinations of the various features described hereinabove, as well as variations and modifications thereof which would occur to persons skilled in the art upon reading the foregoing description and which are not disclosed in the prior art.

1. A medical device, comprising:
a housing;
an annular fixture contained in the housing and having a central opening;
a needle having a proximal end contained within the central opening of the annular fixture and a distal end protruding out of the housing;
a motion assembly, which is configured to move the needle longitudinally back and forth relative to the annular fixture and the housing; and
a flexible diaphragm comprising a peripheral part that seals externally around the annular fixture and an inner sleeve that seals around the proximal end of the needle, whereby movement of the needle back and forth causes the sleeve to evert and invert within the central opening while maintaining a vacuum seal between the annular fixture and the needle.

2. The device according to claim 1, further comprising a tubular casing having a proximal tip coupled with the housing and a distal tip configured for insertion into a body of a patient, with a window opening radially in the distal tip of the tubular casing, wherein the needle is contained within the tubular casing and mounted so that the distal end of the needle moves back and forth across the window, thereby enabling tissue to be cut within the window.

3. The device according to claim 1, wherein the motion assembly comprises a motor, which is configured to move the needle back and forth in a reciprocating motion.

4. The device according to claim 1, wherein the needle contains a lumen, which opens at both the distal and proximal ends, and wherein the device comprises an aspiration tube, which is coupled in fluid communication with the proximal end of the needle so as to provide suction via the lumen through the distal end of the needle.

5. The device according to claim 4, wherein the peripheral part of the diaphragm seals externally around a distal side of the annular fixture, while the aspiration tube is sealed to a proximal side of the annular fixture, thereby creating a sealed chamber within the central opening of the annular fixture, such that the proximal end of the needle moves longitudinally within the sealed chamber relative to the aspiration tube.

6. A method for operating a device, comprising:
providing a medical device, wherein the medical device comprises:
a housing,
an annular fixture contained in the housing and having a central opening,
a needle having a proximal end contained within the central opening of the annular fixture and a distal end protruding out of the housing,
a motion assembly, which is configured to move the needle longitudinally back and forth relative to the annular fixture and the housing, and
a flexible diaphragm comprising a peripheral part that seals externally around the annular fixture and an inner sleeve that seals around the proximal end of the needle; and
moving the needle back and forth within the annular fixture thereby causing the sleeve to evert and invert within the central opening while maintaining a vacuum seal between the annular fixture and the needle.

7. The method according to claim 6, further comprising a tubular casing having a proximal tip coupled with the housing and a distal tip configured for insertion into a body of a patient, with a window opening radially in the distal tip of the tubular casing, wherein the needle is contained within the tubular casing and mounted so that the distal end of the needle moves back and forth across the window, thereby enabling tissue to be cut within the window.

8. The method according to claim 6, wherein the motion assembly comprises a motor, which is configured to move the needle back and forth in a reciprocating motion.

9. The method according to claim 6, wherein the needle contains a lumen, which opens at both the distal and proximal ends, and wherein the device comprises an aspiration tube, which is coupled in fluid communication with the proximal end of the needle so as to provide suction via the lumen through the distal end of the needle.

10. The method according to claim 9, wherein the peripheral part of the diaphragm seals externally around a distal side of the annular fixture, while the aspiration tube is sealed to a proximal side of the annular fixture, thereby creating a sealed chamber within the central opening of the annular fixture, such that the proximal end of the needle moves longitudinally within the sealed chamber relative to the aspiration tube.
